(19) United States
(12) Patent Application Publication
(10) Pub. No.: US 2026/0272354 A1
(43) Pub. Date: Sep. 17, 2026

(54) MUSCLE CONDITION ESTIMATION DEVICE AND METHOD FOR ESTIMATING MUSCLE CONDITION

(71) Applicant: NATIONAL INSTITUTE OF ADVANCED INDUSTRIAL SCIENCE AND TECHNOLOGY, TOKYO (JP)

(72) Inventors: Akihiko MURAI, Ibaraki (JP); Shusuke KANAZAWA, Ibaraki (JP); Ko AYUSAWA, Ibaraki (JP); Souhei WASHINO, Ibaraki (JP); Masaaki MOCHIMARU, Ibaraki (JP)

(21) Appl. No.: 19/670,355

(22) Filed: May 7, 2026

Related U.S. Application Data
(62) Division of application No. 18/686,642, filed on Aug. 19, 2022.

(30) Foreign Application Priority Data
Aug. 25, 2021 (JP) 2021-136871

(51) Int. Cl. A61B 5/22; A61B 5/107; A61B 5/389

(52) U.S. Cl. CPC A61B 5/22; A61B 5/107; A61B 5/389

(57) ABSTRACT

An apparatus and method capable of accurately estimating a participant's muscle state are provided. In order to solve this problem, a muscle state estimation device 1 for estimating the state of a participant's muscles is provided. The muscle state estimation device 1 comprises a circumference sensor 2 that measures the circumference of the muscle, a myopotential sensor 3 that measures the electric potential generated by the activity of the muscle and a muscle state estimation unit 4 that calculates the muscle state such that an evaluation function, of which variables are the deviation of the circumference measured by the circumference sensor 2 from the circumference model that defines the relationship between the muscle state and the circumference and the deviation of the electric potential measured by the myopotential sensor 3 from the muscle tension model that defines the relationship between the muscle state and the electric potential, is minimized.

### TECHNICAL FIELD

This disclosure relates to technology for estimating the state of muscles, hereinafter simply referred to as “muscles”, constituting a body.

### BACKGROUND ART

Various measuring devices and the like have been devised to estimate the state of the body. For example, the following Patent Literature 1 discloses a body information generator 1 that evaluates the usage of muscles of a golf swinging user by detecting changes in the circumference of the biceps brachii muscle of the user.

In addition, the following Patent Literature 2 discloses a device for measuring the grip strength of a participant according to changes in the circumference length of a predetermined part of the participant's arm, and the like.

In addition, the following Patent Document 3 discloses a device, etc., for obtaining the participant's muscle strength based on a musculoskeletal model, from motion data obtained using a motion capture system, myoelectric potentials, and floor reaction force data.

### RELATED-ART LITERATURE

### Patent Literature

Patent Literature 1: JP2017-108871
Patent Literature 2: JP2018-110658
Patent Literature 3: WO2005/122900A1

### SUMMARY OF INVENTION

### Technical Problem

As described above, the body information generating device 1 described in Patent Literature 1 can evaluate the user's muscle usage and the technology described in Patent Literature 2 can measure the participant's grip strength, respectively.

According to the technology described in Patent Literature 3, the participant's muscle strength can be obtained.

However, all of these techniques are not capable of accurately estimating the muscle status of the user or participant.

The purpose of this disclosure is to provide an apparatus and method capable of accurately estimating the state of a participant's muscles.

### Solution to Problem

In order to solve this problem, this disclosure provides a muscle state estimation device for estimating the state of a participant's muscle, comprising a circumference measuring means that measures the circumference of the muscle a myopotential measuring means that measures the electric potential generated by the activity of the muscle and a muscle state evaluation means for that calculates the muscle state such that an evaluation function, of which variables are the deviation of the circumference measured by the circumference measuring means from the circumference model that defines the relationship between the muscle state and the circumference and the deviation of the electric potential measured by the myopotential measuring means from the muscle tension model that defines the relationship between the muscle state and the electric potential, is minimized.

Also in order to solve this problem, this disclosure provides a muscle state estimation device for estimating the state of a participant's muscle, comprising a circumference measuring means that measures the circumference of the muscle a motion state measuring means that measures the motion state of the participant's whole body and a muscle state evaluation means that calculates the muscle state such that an evaluation function, of which variables are the deviation of the circumference measured by the circumference measuring means from the circumference model that defines the relationship between the muscle state and circumference and the deviation of the motion state measured by motion state measuring means from the joint torque model that defines the relationship between the motion state and the joint torque produced in the participant, is minimized.

Also in order to solve this problem, this disclosure provides a muscle state estimation device for estimating the state of a participant's muscle, comprising a circumference measuring means that measures the circumference of the muscle and a muscle state evaluation means that calculates the muscle state such that an evaluation function, of which variables are the deviation of the circumference measured by the circumference measuring means from the circumference model that defines the relationship between the muscle state and circumference and from the learned circumference model constructed by machine learning the correlation between the circumference and the muscle state, is minimized.

Also in order to solve this problem, this disclosure provides a muscle state estimation device for estimating the state of a participant's muscle, comprising a circumference measuring means that measures the circumference of the muscle and a muscle state evaluation means that inputs the measured circumference into a probability circumference model constructed by probability distribution of the entire system using the measured circumference as the observed series and the muscle activity data as the latent variable series with respect to muscle activity data having a correlation with the circumference and the circumference measured by the circumference measuring means, performs maximum likelihood series estimation and calculates the muscle activity data corresponding to input the circumference.

Also in order to solve this problem, this disclosure provides a muscle state estimation method for estimating the state of a participant's muscle, comprising a first step of measuring the circumference of the muscle and the electric potential generated by the activity of the muscle and a second step of calculating the muscle state such that an evaluation function, of which variables are the deviation of the circumference measured in the first step from the circumference model that defines the relationship between the muscle state and the circumference and the deviation of the electric potential measured in the first step from the muscle tension model that defines the relationship between the muscle state and the electric potential, is minimized.

Also in order to solve this problem, this disclosure provides a muscle state estimation method for estimating the state of a participant's muscle, comprising a first step of the circumference of the muscle and the motion state of the participant's whole body and a second step of calculating the muscle state such that an evaluation function, of which variables are the deviation of the circumference measured in the first step from the circumference model that defines the relationship between the muscle state and circumference and the deviation of the motion state measured in the first step from the joint torque model that defines the relationship between the motion state and the joint torque produced in the participant, is minimized.

Also in order to solve this problem, this disclosure provides a muscle state estimation method for estimating the state of a participant's muscle, comprising a first step of measuring the circumference of the muscle and a second step of calculating the muscle state such that an evaluation function, of which variables are the deviation of the circumference measured in the first step from the circumference model that defines the relationship between the muscle state and circumference and from the learned circumference model constructed by machine learning the correlation between the circumference and the muscle state, is minimized.

Also in order to solve this problem, this disclosure provides a muscle state estimation method for estimating the state of a participant's muscle, comprising a first step of measuring circumference of the muscle and a second step of inputting the measured circumference into a probability circumference model constructed by probability distribution of the entire system using the measured circumference as the observed series and the muscle activity data as the latent variable series with respect to muscle activity data having a correlation with the circumference and the circumference measured in the first step, performs maximum likelihood series estimation and calculates the muscle activity data corresponding to input the circumference.

### Advantageous Effects of Invention

According to the present disclosure, it is possible to provide a muscle state estimation device and a muscle state estimation method that can accurately estimate the state of a participant's muscles.

### BRIEF DESCRIPTION OF DRAWINGS

FIG. 1 is a block diagram showing the configuration of the muscle state estimation device 1 according to Embodiment 1 of the present disclosure;

FIG. 2 shows a flowchart showing the muscle state estimation method according to Embodiment 1 of the present disclosure;

FIG. 3 is a block diagram showing the configuration of the muscle state estimation device 10 according to Embodiment 2 of the present disclosure;

FIG. 4 is a flowchart showing the muscle state estimation method according to Embodiment 2 of the present disclosure;

FIG. 5 is a block diagram showing the configuration of the muscle state estimation device 20 according to Embodiment 3 of the present disclosure;

FIG. 6 is a flowchart showing the muscle state estimation method according to Embodiment 3 of the present disclosure;

FIG. 7 is a block diagram showing the configuration of the muscle state estimation device 30 according to Embodiment 4 of the present disclosure; and

FIG. 8 is a flowchart showing the muscle state estimation method according to Embodiment 4 of the present disclosure.

### DESCRIPTION OF EMBODIMENTS

### Embodiment 1

In the following, Embodiment 1 of the disclosure will be explained in detail with reference to the drawings. The same symbols in the figures indicate the same or equivalent parts.

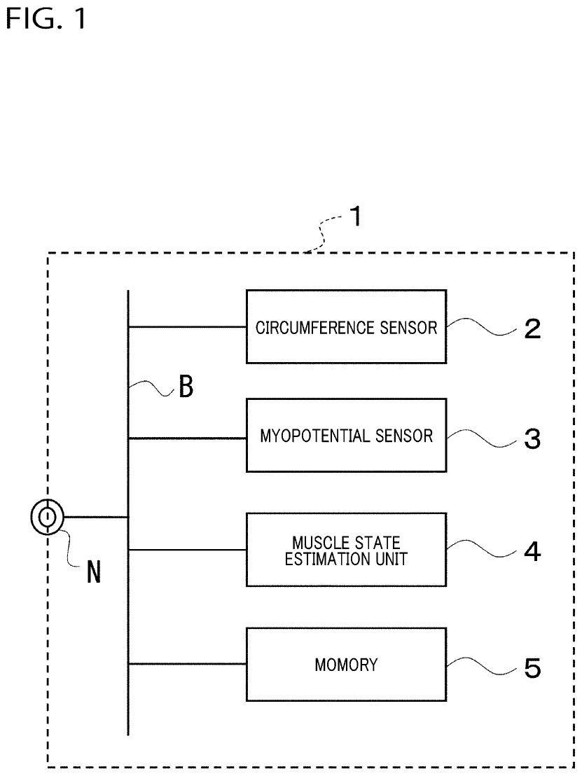

FIG. 1 is a block diagram showing the configuration of the muscle state estimation device 1 according to Embodiment 1 of the present disclosure. As shown in FIG. 1, the muscle state estimation device 1 comprises an input/output node N, a bus B connected to the input/output node N, a circumference sensor 2, a myopotential sensor 3, a muscle state estimation unit 4 and a memory 5, each connected to the bus B.

As the circumference sensor 2, for example, a known wearable device that is worn around a body part such as the upper arm in the form of a band and measures the circumference of the muscle concerned by sensing changes in the length of a material made of stretchable fiber, rubber, or the like can be used.

As the myopotential sensor 3, for example, a commercially available wearable device that includes two electrodes positioned on a muscle run in a body part such as the upper arm and measures the action potential that appears during muscle activity as a potential difference between the above electrodes, namely myopotential, can be used.

The muscle state estimation unit 4 is composed of a central processing unit, CPU, operated by a predetermined program, the operation of which is described below. The program is stored in the memory 5 in advance via the input/output node N and the bus B.

The memory 5 further stores the data measured by the circumference sensor 2 and the myopotential sensor 3 and the results obtained by the muscle state estimation unit 4.

The above results can be displayed, etc., by outputting them to an external monitor, etc., via the bus B and the input/output node N.

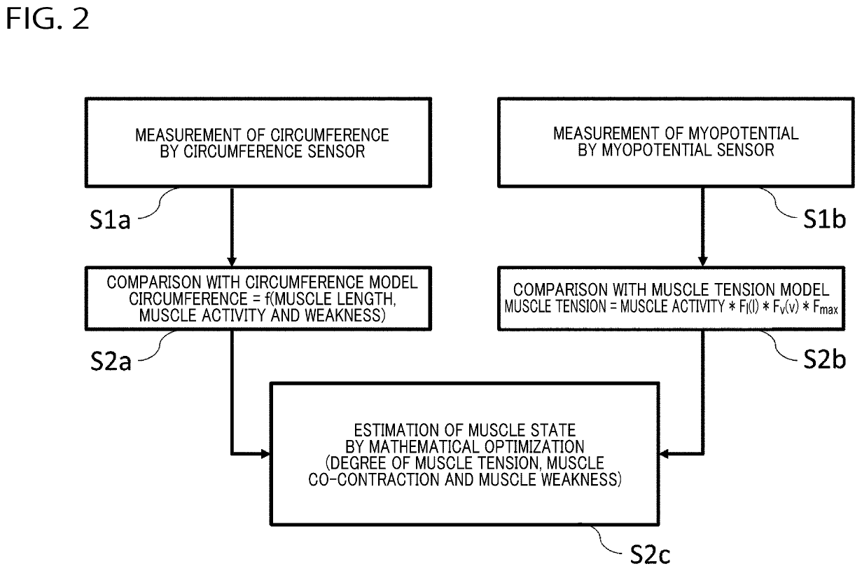

FIG. 2 is a flowchart showing the muscle state estimation method according to Embodiment 1 of the present disclosure. In the following, the muscle state estimation method according to Embodiment 1 of the present disclosure will be explained using the muscle state estimation device 1 shown in FIG. 1 as an example. However, it goes without saying that this method is not limited to the use of the muscle state estimation device 1, but can also be implemented by other means.

In the step S1a, a circumference sensor 2 is used to measure the circumference of a muscle of the upper arm or the like, which constitutes a body part, and in the step S1b, a myopotential sensor 3 is used to measure the myopotential in the above muscle.

Next, in the step S2a, the muscle state estimation unit 4 compares the circumference measured in the step S1a with a circumference model prepared in advance. This circumference model is stored in memory the 5 or an external device such as a cloud, not shown in the figure.

The circumferential model is a model in which the circumferential length is represented by a function f of the muscle length and the degree of muscle activity or weakness, and is modeled based on the fact that the volume of the muscle is constant, that the muscle is deformed so that the cross-sectional area of the muscle belly portion increases due to muscle activity, that the elasticity of the muscle decreases due to weakness and that the muscle is deformed passively in response to external forces such as elasticity by the circumferential sensor.

Specifically, the known model described in Murai, A., Hong, Q. Y., Yamane, K., & Hodgins, J. K., 2017, Dynamic skin deformation simulation using musculoskeletal model and soft tissue dynamics, Computational Visual Media, 3(1), pp. 49-60, hereinafter referred to as the “muscle deformation model”, can be used as the first example of a circumference model.

The known model described in YAMAMURA, N., Alves, J. L., ODA, T., KINUGASA, R., & TAKAGI, S., 2014, Effect of tendon stiffness on the generated force at the Achilles tendon-3D finite element simulation of a human triceps surae muscle during isometric contraction, Journal of Biomechanical Science and Engineering, 9(3), pp. 13-00294, hereinafter referred to as “FEM model”, which includes a mechanics simulation such as the finite element method can also be used as the second example.

While the muscle deformation model and the FEM model described above only represent the relationship between the positive muscle activity and the muscle deformation, the circumferential model is further defined as the relationship between the circumference of the muscle and the state of muscle weakness. In other words, this circumference model can take into account the degree of muscle softening and the accompanying passive deformation, by adding the circumference represented as a function of the degree of weakness and the inertia or external force to the above function f as a correction term which represents the negative muscle activity indicating the physical softness of the muscle caused by weakness due to external force intervention in neurally controlled relaxation or stretching, etc.

The muscle state estimation unit 4 obtains the circumference from the previously obtained measurements and estimated values with respect to the muscle length, which indicates the shape of the muscle, and the degree of muscle activity and weakness, which indicates the shape characteristics and stiffness of the muscle, and compares it with the circumference actually measured in step S1a, as described above.

On the other hand, in step S2b, the muscle state estimation unit 4 compares the myopotential measured in step S1b with a muscle tension model prepared in advance. This muscle tension model is also stored in an external device, not shown in the figure, such as memory 5 or the cloud.

The muscle tension model is a model that represents muscle tension as the product of muscle activity evaluated by myopotentials, the function F₁(l) of muscle length and muscle force, the function Fᵥ(v) of muscle length change rate and muscle force, and the maximum muscle tension Fₘₐₓ, and is modeled to have muscle activity and muscle length as variables.

Specifically, for example, the well-known hill-type muscle model can be used as a model of muscle tension.

Then, the muscle state estimation unit 4 obtains the myopotential from the previously obtained measurements and estimated values with respect to the muscle length, which indicates the shape of the muscle, and compares it with the myopotential actually measured in step S1b as described above.

Next, in step S2c, the muscle state estimation unit 4 calculates and estimates the participant's muscle state, such as muscle tension, muscle co-contraction and degree of muscle weakness, so as to be optimized mathematically, namely, such that the evaluation function, of which variables are the deviation of the above circumference measured by the circumference sensor 2 from the circumference model that defines the relationship between the muscle state and the circumference and the deviation of the electric potential measured by the myopotential sensor 3 from the muscle tension model that defines the relationship between the muscle state and the above electric potential, is minimized.

Specifically, the muscle state estimation unit 4, for example, calculates the degree of muscle tension, muscle co-contraction and muscle weakness such that the evaluation function Z1 shown in the following equation (1) is minimized.

$$\begin{matrix}[ equation 1 ] & \\ Z 1 = C 1 ( [ MEASURED CIRCUMFERENCE ] - [ CIRCUMFERENCE CALCULATED FROM CIRCUMFERENCE MODEL ] ) + C 2 ( [ MEASURED MUSCLE ACTIVITY ] - [ MUSCLE ACTIVITY CALCULATED FROM MUSCLE TENSION MODEL ] ) + C 3 ( [ FUNCTION OF MUSCLE ACTIVITY AND MOTION BASED ON KNOWLEDGE ON MOTION AND PHYSIOLOGY ] ) & ( 1 )\end{matrix}$$

It should be noted that C1, C2 and C3 in equation (1) represent different constants.

In addition, “functions of muscle activity and movement based on exercise and physiological knowledge” in the third term on the right side of equation (1) means functions of muscle activity and movement based on general exercise and physiological knowledge that represents “small co-contraction during quick movement,” “large co-contraction during precise manipulation” and “reduction of joint compression force such as intervertebral disc pressure.” One example is the function disclosed in JP2019-5344.

In the above, for example, in the case of the upper arm, the muscle state of each muscle, such as the degree of muscle tension, muscle co-contraction and muscle weakness, can be estimated if a model consisting of multiple muscles such as the biceps brachii and triceps brachii is considered and if the above optimization calculation is performed from the circumference and muscle activity obtained from the circumference sensor 2 and the multiple myopotential sensors 3 corresponding to the number of muscles in the above model. In particular, even when the muscle activity is 0, the degree of muscle weakness can be estimated because the optimization can be performed considering the inequality constraint conditions for negative muscle circumference and muscle activity.

According to the muscle state estimation device 1 and the muscle state estimation method shown in FIG. 2 of Embodiment 1 of the disclosure as described above, by making the co-contraction including both antagonistic muscles the target of measurement by the circumference sensor 2, it is possible to quantitatively estimate not only the active deformation of the muscle but also the passive deformation of the muscle, i.e., the degree of weakness, which cannot be obtained by conventional kinematic analysis, while avoiding the difficulty of measuring only the muscle activity of one side.

In addition, the muscle state estimation device 1 can be configured as a wearable device because the measurement target can be limited to a part of the participant's body as described above.

### Embodiment 2

In the following, Embodiment 2 according to the disclosure will be described. The description of the parts in common with Embodiment 1 above will be omitted and mainly the different parts will be described.

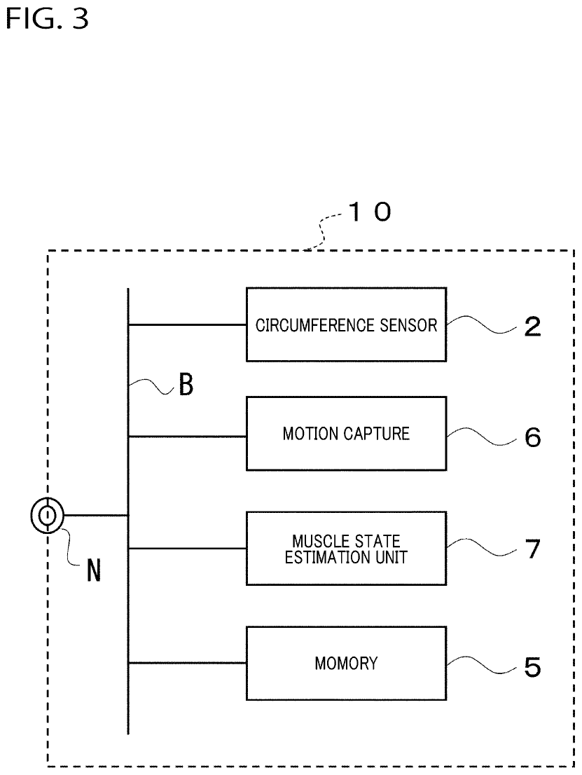

FIG. 3 is a block diagram showing the configuration of the muscle state estimation device 10 according to Embodiment 2 of the present disclosure. As shown in FIG. 3, the muscle state estimation device 10 according to Embodiment 2 comprises the motion capture 6 instead of the myopotential sensor 3 in the muscle state estimation device 1 according to Embodiment 1.

Known technologies to measure the participant's whole body movement by optical or video method can be used as the motion capture 6.

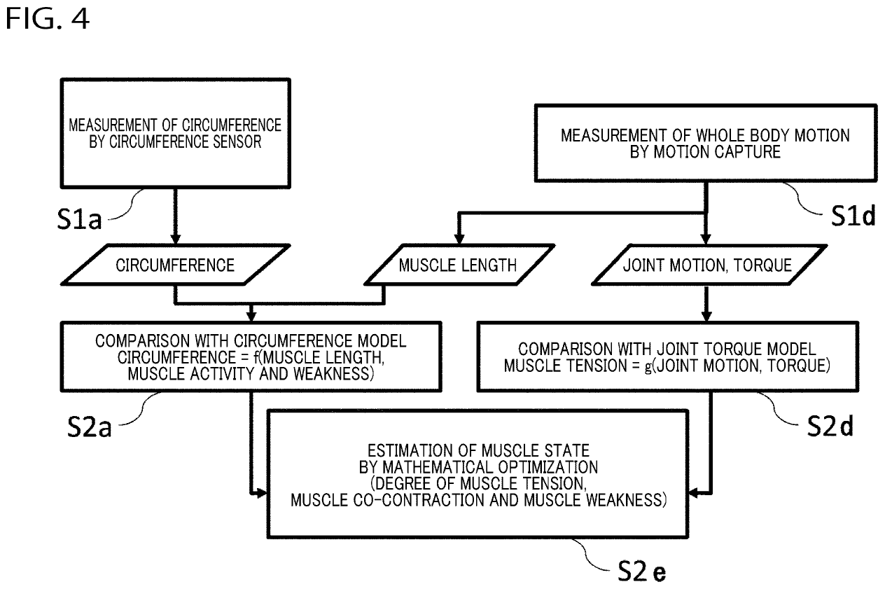

FIG. 4 is a flowchart showing the muscle state estimation method according to Embodiment 2 of the present disclosure. In the following, the muscle state estimation method according to Embodiment 2 of the present disclosure will be explained using the muscle state estimation device 10 shown in FIG. 3 as an example. However, it goes without saying that this method is not limited to using the muscle state estimation device 10 and can also be implemented by other means.

In step S1d, the motion state of the participant's whole body, i.e., muscle length and joint torque, for example, are measured by the motion capture 6. Muscle length and joint torque are calculated by inverse kinematics calculation using a musculoskeletal model by the muscle state estimation unit 7.

The musculoskeletal model may be, for example, the well-known model described in the above Patent literature 3. This model is stored in an external device such as memory 5 or cloud, not shown in the figure.

Next, in step S2a, the muscle state estimation unit 7 compares the circumference measured in step S1a and the muscle length obtained in step S1d with the circumference model prepared in advance as described above.

On the other hand, in step S2d, the muscle state estimation unit 7 compares the joint torque obtained in step S1b with the joint torque model prepared in advance.

The known model that distributes joint torque to muscle tension based on joint motion using the function g shown in FIG. 4 can be used as the above joint torque model. One example is the model described in Murai, A., Takeichi, K., Miyatake, T., & Nakamura, Y., 2014, September, Musculoskeletal modeling and physiological validation, In 2014 IEEE International Workshop on Advanced Robotics and its Social Impacts, pp. 108-113, IEEE.

Then, in step S2e, the muscle state estimation unit 7 calculates and estimates the participant's muscle state, such as muscle tension, muscle co-contraction, and degree of muscle weakness, so as to be optimized mathematically, namely, such that the evaluation function, of which variables are the deviation of the above circumference measured by the circumference sensor 2 from the circumference model that defines the relationship between the muscle state and the circumference and the deviation of the above motion state measured by the motion capture 6 from the above joint torque model that defines the relationship between the motion state and the joint torque produced in the participant, is minimized.

Specifically, the muscle state estimation unit 4, for example, calculates and estimates the degree of muscle tension, muscle co-contraction and muscle weakness of the participant such that the evaluation function Z2 shown in the following equation (2) is minimized.

### [Equation 2]

$$\begin{matrix}[ equation 2 ] & \\ Z 2 = C 21 ( [ MEASURED CIRCUMFERENCE ] - [ CIRCUMFERENCE CALCULATED FROM CIRCUMFERENCE MODEL ] ) + C 22 ( [ MEASURED JOINT TORQUE ] - [ JOINT TORQUE CALCULATED FROM JOINT TORQUE MODEL ] ) + C 23 ( [ FUNCTION OF MUSCLE ACTIVITY AND MOTION BASED ON KNOWLEDGE ON MOTION AND PHYSIOLOGY ] ) & ( 2 )\end{matrix}$$

It should be noted that C21 C22, and C23 in equation (2) represent different constants.

The muscle state estimation device 10 according to Embodiment 2 further comprises the myopotential sensor 3 shown in FIG. 1. Then, the muscle state estimation unit 7 adds the deviation of the electric potential measured by the myopotential sensor 3 from the muscle tension model according to Embodiment 1 that defines the relationship between the muscle state and the above electric potential to the variables of the evaluation function Z2. Thus, the muscle state can be estimated more precisely by calculating the muscle state such that the evaluation function Z2 is minimized.

As described above, according to the muscle state estimation device 10 according to Embodiment 2 of the present disclosure and the muscle state estimation method shown in FIG. 4, in addition to achieving the same effects as those of the aforementioned muscle state estimation device 1 and the muscle state estimation method according to Embodiment 1, by measuring the physical quantity of muscle circumference instead of myopotential, the effect of impedance change between the electrode and the skin can be avoided, thus, as a result, stable measurement over a long period of time can be realized.

### Embodiment 3

In the following, the parts in common with Embodiments 1 and 2 above will be omitted, and the different parts will be explained mainly.

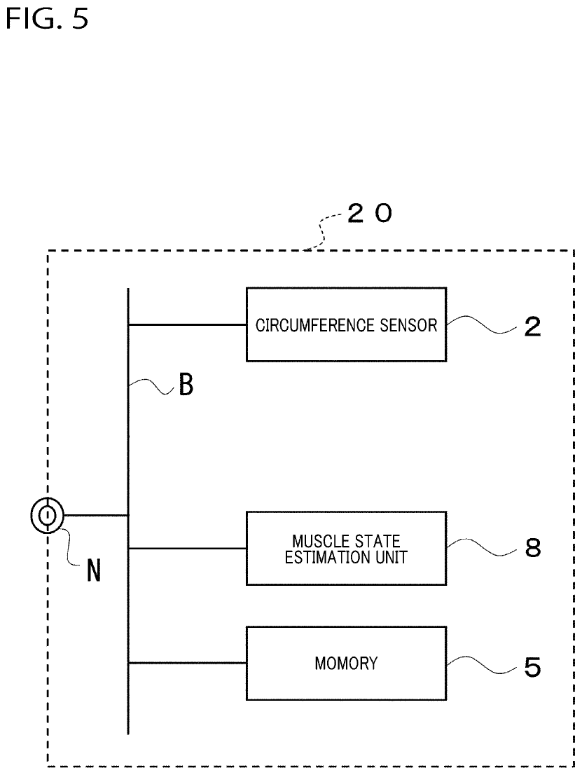

FIG. 5 is a block diagram showing the configuration of the muscle state estimation device 20 according to Embodiment 3. As shown in FIG. 5, the muscle state estimation device 20 according to Embodiment 3 has a configuration in which the myopotential sensor 3 is excluded from the muscle state estimation device 1 according to Embodiment 1.

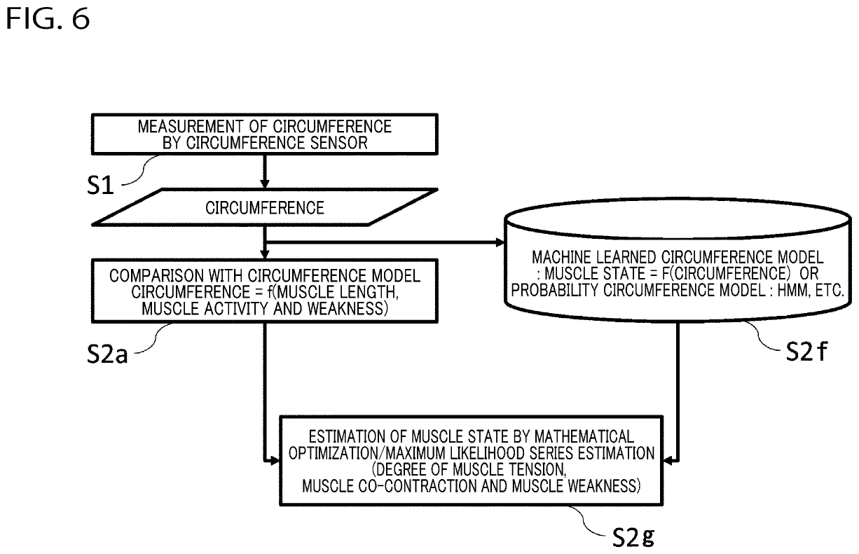

FIG. 6 is a flowchart showing the muscle state estimation method according to Embodiment 3. In the following, the muscle state estimation method according to Embodiment 3 of the present disclosure will be described using the muscle state estimation device 20 shown in FIG. 5 as an example. However, it goes without saying that this method is not limited to the case of using the muscle state estimation device 20 and can be implemented by other means.

When the data on circumference, myopotential, whole body movement and muscle state, i.e., muscle activity and weakness, obtained through the implementation of Embodiments 1 or 2, etc. above are accumulated, a pattern of muscle state, i.e., muscle activity and weakness, correspond to a certain pattern of circumference and a correlation model can be constructed between the pattern of muscle state and the pattern of circumference. Therefore, by utilizing this correlation model, it is possible to estimate the data of the muscle state from the circumference data.

That is, in step S2f, the muscle state estimation unit 8 compares, i.e., inputs, the circumference measured in step S1 with the learned circumference model or the probability circumference model. The learned circumference model is constructed by machine learning the correlation F between the changes in the muscle circumference, circumference, and muscle activity state, muscle state, using circumference and muscle activity data obtained independently as described above, as teacher data. On the other hand, the probability circumference model is constructed by obtaining the probability distribution of the entire system, for the circumference data and muscle activity data obtained independently, using the circumference data as the observed series and the muscle activity data as the latent variable series in a probability transition model represented by a hidden Markov model, HMM. The above probability transition model refers to any model that expresses the relationship between the multiple time series data, either synchronous or asynchronous, and that estimates or generates other time series data from some time series data, including regression models of time series and other statistical models that are not probability models.

The learned circumference model or the probability circumference model is stored in memory 5 or an external device such as a cloud, not shown in the figure.

Next, in step S2g, the muscle state estimation unit 8 calculates and estimates the participant's muscle state so as to be optimized mathematically, namely, such that the evaluation function, of which variables are the deviation of the above circumference measured by the circumference sensor 2 from the circumference model that defines the relationship between the muscle state and the circumference and from the above learned model, is minimized. On the other hand, when the above probability circumference model is used, the muscle state estimation unit 8 calculates the muscle activity data, which is a latent variable series that generates a specific observation series by maximum likelihood series estimation when the circumference is observed.

Also according to the aforementioned muscle state estimation device 20 according to Embodiment 3 of the present disclosure and the muscle state estimation method shown in FIG. 6, in the same way as the muscle state estimation device 10 and the muscle state estimation method according to Embodiment 2 above, myopotentials are not measured, therefore, the effect of impedance change between the electrodes and the skin can be avoided, thus, as a result, stable measurement over a long period of time can be realized.

### Embodiment 4

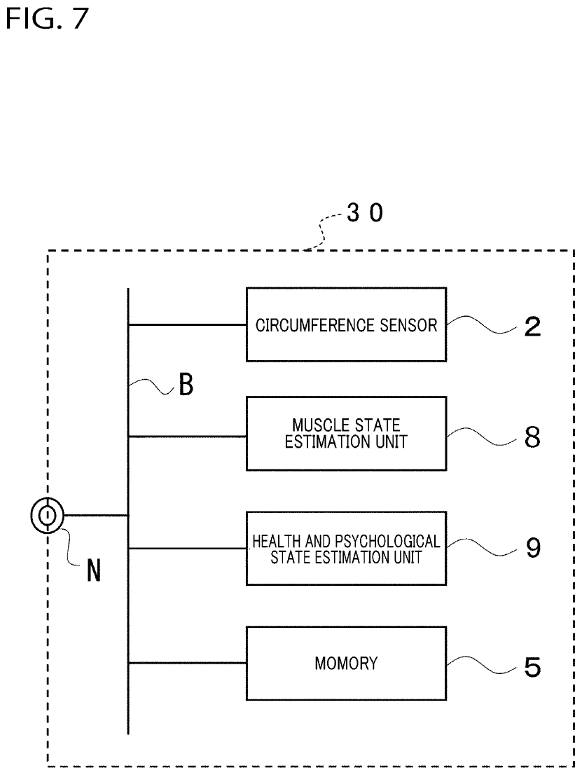

FIG. 7 is a block diagram showing the configuration of the muscle state estimation device 30 according to Embodiment 4 of the disclosure. As shown in FIG. 7, the muscle state estimation device 30 according to Embodiment 4 has a similar configuration to the muscle state estimation device 20 according to Embodiment 3 shown in FIG. 5, but differs in that it further comprises a health and psychological state estimation unit 9 connected to the bus B.

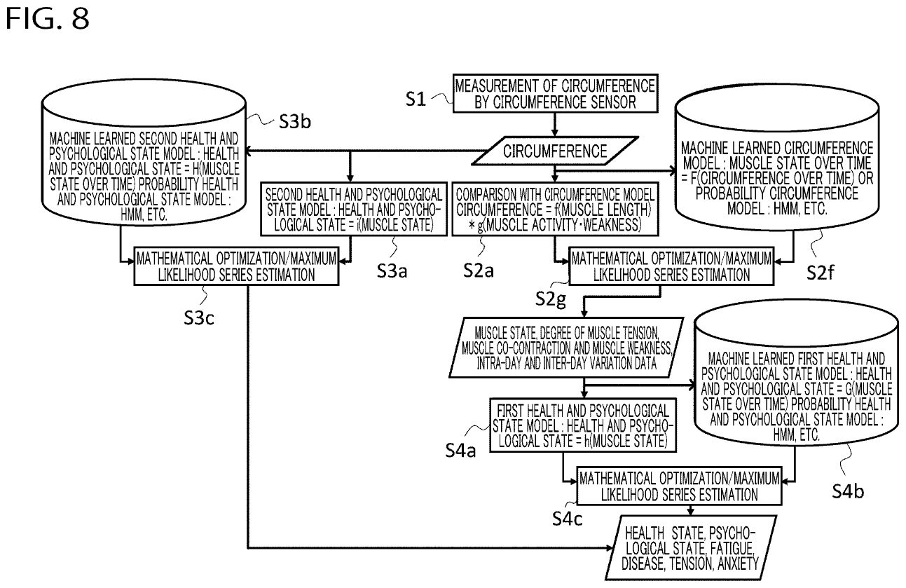

FIG. 8 is a flowchart showing the muscle state estimation method according to Embodiment 4. In the following, the muscle state estimation method according to Embodiment 4 will be explained with reference to FIG. 8. The description of the parts in common with Forms 1 to 3 above is omitted, and the different parts are mainly explained.

In the following, we will explain the muscle state estimation method according to Embodiment 4 by implementing muscle state estimation device 30 shown in FIG. 7 because the method can be implemented using the muscle state estimation device 30. However, it goes without saying that it can also be implemented by other means.

When data on circumference, myopotential, whole body movement and muscle state, i.e., muscle activity and weakness, measured in the manner described in Embodiments 1 and 2 above, and data on health conditions such as fatigue and disease, and psychological states such as tension and anxiety measured simultaneously with all or part of these at the same or different granularity are accumulated, a pattern of the above health and psychological states corresponds to a certain circumference pattern or a circumference pattern for a long time such as intra-day or inter-day variation, therefore, a correlation model can be constructed between the above health and the psychological states pattern and the circumference pattern. Therefore, by utilizing this correlation model, it is possible to estimate health and psychological state data from circumference data.

Specifically, in step S3a, the health and psychological state estimation unit 9 compares the muscle state calculated by the muscle state estimation unit 8 with the second health and psychological state model that represents the above health and psychological states as a function i of muscle state.

In step S3b, the health and psychological state estimation unit 9 also compares the muscle states calculated over time by the muscle state estimation unit 8 with the muscle state calculated by the second learned health and psychological model that is machine-learned by correlating these H using the corresponding data on the above health and psychological states as teacher data or the probabilistic health and psychological model based on a probabilistic transition model such as a hidden Markov model (HMM).

Similarly, in step S4a, the health and psychological state estimation unit 9 compares the intra-day and inter-day variation data on the muscle state and the degree of muscle tension, muscle co-contraction and muscle weakness calculated by the muscle state estimation unit 8 based on the above circumference with the first health and psychological state model representing health and psychological states as a function of muscle state h.

In step S4b, the health and psychological state estimation unit 9 compares the muscle states calculated over time by the muscle state estimation unit 8 with the muscle state calculated by the first learned health and psychological model that is machine-learned by correlating these G using the corresponding data on the above health and psychological states as teacher data or the probabilistic health and psychological model based on a probabilistic transition model such as a hidden Markov model (HMM).

Then, in steps S3c and S4c, the health and psychological state estimation unit 9 estimates the health and psychological states of the participant, such as fatigue, disease, tension, and anxiety so as to be optimized mathematically, namely, such that the evaluation function, of which variables are the deviation from the above second and first health and psychological model and the above second and first learned health and psychological model, is minimized or so as be performed maximum likelihood series estimation using the above probabilistic health and psychological model.

The above probability transition model refers to any model that expresses the relationship between the multiple time series data, either synchronous or asynchronous, and that estimates or generates other time series data from some time series data, including regression models of time series and other statistical models that are not probability models, similarly with Embodiment 3.

In the above, the health and psychological state estimation unit 9 may be integrated with the muscle state estimation unit 8 to estimate the health and psychological state of the participant by directly calculating data related to the health and psychological state from the circumference without involving the muscle state.

According to the muscle state estimation device 30 according to Embodiment 4 of this disclosure and the muscle state estimation method shown in FIG. 8 as described above, the health and psychological state of the participant can be quantitatively estimated by the simple method of measuring the circumference of the muscle with the circumference sensor 2.

### REFERENCE SIGNS LIST

1, 10, 20, 30 muscle state estimation device,
2 circumference sensor,
3 myopotential sensor,
4, 7, 8 muscle state estimation unit,
6 motion capture,
9 health and psychological state estimation unit.

1. A muscle state estimation device for estimating the state of a participant's muscle, comprising:
a circumference measuring means that measures the circumference of the muscle;
a motion state measuring means that measures the motion state of the participant's whole body; and
a muscle state evaluation means that calculates the muscle state such that an evaluation function, of which variables are the deviation of the circumference measured by the circumference measuring means from the circumference model that defines the relationship between the muscle state and circumference and the deviation of the motion state measured by motion state measuring means from the joint torque model that defines the relationship between the motion state and the joint torque produced in the participant, is minimized.

2. The muscle state estimation device according to claim 1, further comprising a myopotential measuring means that measures the electric potential generated by the activity of the muscle,
wherein the muscle state estimation means further adds the deviation of the electric potential measured by the myopotential measuring means from the muscle tension model that defines the relationship between the muscle state and the electric potential to the variables of the evaluation function and calculates the muscle state such that an evaluation function is minimized.

3. A muscle state estimation method for estimating the state of a participant's muscle, comprising:
a first step of the circumference of the muscle and the motion state of the participant's whole body; and
a second step of calculating the muscle state such that an evaluation function, of which variables are the deviation of the circumference measured in the first step from the circumference model that defines the relationship between the muscle state and circumference and the deviation of the motion state measured in the first step from the joint torque model that defines the relationship between the motion state and the joint torque produced in the participant, is minimized.

4. The muscle state estimation method according to claim 3,
wherein the first step further measuring the electric potential generated by the activity of the muscle and
wherein the second step adding the deviation of the electric potential measured in the first step from the muscle tension model that defines the relationship between the muscle state and the electric potential to the variables of the evaluation function and calculating the muscle state such that an evaluation function is minimized.
